(19) United States
(12) Patent Application Publication
(10) Pub. No.: US 2026/0258456 A1
(43) Pub. Date: Sep. 3, 2026

(54) Method for Synthesizing Lactic Acid Monomers Using Sucrose as Raw Material

(71) Applicant: Tianjin University of Science and Technology, Tianjin (CN)

(72) Inventors: Zhengxiang Wang, Tianjin (CN); Dandan Niu, Tianjin (CN); Meng Wang, Tianjin (CN)

(21) Appl. No.: 19/458,995

(22) Filed: Jan. 26, 2026

(30) Foreign Application Priority Data
Mar. 3, 2025 (CN) 202510238722.9

(51) Int. Cl. C12P 7/56; C12N 15/70

(52) U.S. Cl. CPC C12P 7/56; C12N 15/70

(57) ABSTRACT

Provided is a method for synthesizing lactic acid monomers using sucrose as raw material. Through site-directed gene replacement genetic recombination technology, the method modifies the promoter sequence of the sucrose metabolism operon in the production strain, enabling it to maintain high-level expression throughout the entire production process of lactic acid monomers. The strain exhibits significant ability to efficiently catabolize sucrose for synthesizing lactic acid monomers (D-lactic acid or L-lactic acid) with high optical purity and high chemical purity. Fermentation levels of D-lactic acid and L-lactic acid for strains DSAPW5 and LSAPW5 at 25~50° C. for 28~30 hours can reach 158.58 g/L and 159.65 g/L, respectively, with optical purities of 99.94% and 99.95%. The present disclosure can be applied to the industrial production of lactic acid monomers using sucrose or sucrose-containing raw materials.

### CROSS REFERENCE TO RELATED APPLICATION

This application claims priority to Chinese Patent Application No. 202510238722.9, filed with the China National Intellectual Property Administration on Mar. 3, 2025, entitled “method for synthesizing lactic acid monomers using sucrose as raw material”, the entire contents of which are incorporated herein by reference.

### Reference to Sequence Listing

This application includes a Sequence Listing filed electronically as an XML file named “xlb-PCN1241253-251226.xml”, created on Dec. 30, 2025, with a size of 67,066 bytes. The Sequence Listing is incorporated herein by reference.

### TECHNICAL FIELD

The present disclosure relates to the fields of fermentation engineering and genetic engineering technology, and specifically to a method for synthesizing lactic acid monomers using sucrose as a raw material.

### BACKGROUND

Lactic acid, also known as α-hydroxypropionic acid with the molecular formula C₂H₅OCOOH, is widely used in food, feed, and pharmaceutical additives. In addition, lactic acid monomers with high optical and chemical purity can serve as precursors for biodegradable polymers such as polylactic acid (PLA). Currently, the large-scale production of lactic acid monomers (D-lactic acid or L-lactic acid) through biological metabolism of metabolically engineered bacteria (mainly metabolically engineered Escherichia coli) using glucose (starch) as a raw material is the main production method of lactic acid monomers.

With the expected increase in demand for lactic acid monomers in PLA production, finding stable large-scale production raw materials is crucial for its sustainable development (Zhengxiang Wang. China's Polylactic Acid Industry: Current Status and Development Strategies. Strategic Study of CAE, 2021, 23(6): 155-166). Against this background, sucrose has emerged as a feasible and sustainable chemical production raw material, highlighting its importance in biological manufacturing processes. However, most of the commonly used starting strains for the biological manufacturing of lactic acid monomers-Escherichia coli-cannot directly utilize sucrose (Jahreis, K., et al. Adaptation of sucrose metabolism in the Escherichia coli wild-type strain EC3132. Journal of Bacteriology, 2002, 184(19): 5307-5316), or although they can utilize it, the utilization efficiency is low, lacking industrial application value.

Several studies have been conducted to address the low efficiency of Escherichia coli in utilizing sucrose. Knocking out the cscR gene encoding the negative regulator of sucrose metabolism in strains capable of utilizing sucrose can improve strain growth (Arifin, Y., et al. Deletion of cscR in Escherichia coli W improves growth and poly-3-hydroxybutyrate (PHB) production from sucrose in fed batch culture. Journal of Biotechnology, 2011, 156(4): 275-278). Furthermore, heterologously expressing sucrose transport and hydrolysis-related genes cscA (encoding invertase), cscK (encoding fructokinase), and cscB (encoding sucrose transporter) from sucrose-utilizing Escherichia coli W strains into Escherichia coli strains that cannot metabolize sucrose can endow the recombinant strains with sucrose metabolism ability (Bruschi, M., et al. A transferable sucrose utilization approach for non-sucrose-utilizing Escherichia coli strains. Biotechnology Advances, 2012, 30(5): 1001-1010; Carruthers, D. N., et al. Random chromosomal integration and screening yields E. coli K-12 derivatives capable of efficient sucrose utilization. ACS Synth Biol, 2020, 9(12): 3311-3321). However, their sucrose metabolism efficiency is still low, and they also lack industrial application value. Research reports show that the recombinant Escherichia coli HBUT-L obtained through recombinant technology can metabolize sucrose to produce L-lactic acid after 96 hours of fermentation, indicating that synthesizing lactic acid monomers using sucrose as a raw material is feasible. However, the conversion rate is only 74.0%, the yield is only 60 g/L, and the production intensity is only 0.389 g/(L·h) (Jinfang Zhao et al. Study on L-lactic acid production by Escherichia coli engineering strain using sugarcane molasses as fermentation substrate. Hubei Agricultural Sciences, 2016).

To solve the problem that Escherichia coli cannot effectively metabolize sucrose into lactic acid monomers, adding invertase to hydrolyze sucrose into glucose and fructose in the culture system, and adopting simultaneous saccharification and fermentation can realize lactic acid production from sucrose (Huayu Zhang et al. Simultaneous saccharification and fermentation of sucrose for lactic acid monomer production. Sugarcane and Canesugar, 2021, 50(4): 77-84; Zhengxiang Wang, Kangming Tian, Dandan Niu, Fuping Lu. Chinese Invention Patent, Application No. 202011610612.4, Application Date: Dec. 30, 2020). Heterologously expressing invertase in lactic acid monomer-producing strains to endow them with the ability to hydrolyze sucrose, converting sucrose into glucose and fructose first, which are then metabolized and synthesized into lactic acid monomers by the strains, is also an effective method for biological conversion of sucrose into lactic acid monomers (Zhengxiang Wang, KangmingTian, Dandan Niu, Fuping Lu. Chinese Invention Patent, application No. 202011610612.4, Application Date: Dec. 30, 2020).

To enable Escherichia coli to directly utilize sucrose and perform efficient and simple biological fermentation of lactic acid monomers under anaerobic conditions, the present disclosure creatively screens promoters capable of initiating gene transcription under anaerobic conditions (i.e., so-called anaerobic promoters) based on genome-wide transcriptional profile analysis, on the basis of clarifying that the activation of the initial sucrose metabolism process in Escherichia coli is an aerobic behavior that cannot meet the anaerobic requirements necessary for lactic acid monomer biosynthesis. Then, gene replacement technology is used to replace the original promoter of the initial sucrose metabolism gene in the strain with an anaerobic promoter. The obtained new strain can efficiently complete aerobic metabolic growth on sucrose, and efficiently initiate anaerobic sucrose metabolism under the established new fermentation process, thereby realizing efficient biological synthesis of lactic acid monomers from sucrose. It can be applied to the industrial production of lactic acid monomers using sucrose or sucrose-containing raw materials.

### SUMMARY OF THE INVENTION

The purpose of the present disclosure is to genetically modify the promoter mediating sucrose metabolism in the genome of lactic acid monomer-producing strains through site-directed gene replacement genetic recombination technology. The original promoter is replaced with a promoter that can still initiate high-level transcription under anaerobic acid-producing conditions, to obtain new strains with high-intensity sucrose metabolism ability and efficient synthesis of lactic acid monomers. Thereby, the efficiency of lactic acid monomer production from sucrose raw materials is improved, the production process is significantly simplified, and the production cost is reduced.

To achieve the above purpose, the present disclosure adopts the following technical route:

One of the technical solutions provided by the present disclosure is a method for improving the ability of Escherichia coli to synthesize lactic acid monomers using sucrose, which replaces the promoter of the sucrose metabolism operon cscAKB in the genome of the lactic acid monomer-producing strain with an anaerobic promoter;
further, said anaerobic promoters include but are not limited to: P_{gapA}, P_{gadA}, P_{pflB}, Pₑₙₒ, P_{cysK}, P_{glnA}, P_{pgk}, P_{manX}, P_{adhE}, P_{pfkA}, P_{acnA}, P_{hchA}, P_{agp}, P_{serA}, Pₘₑₗₐ, P_{tpiA}, P_{pykA}, P_{fruK}, P_{ackA}, P_{yqhD}, etc.;
preferably, said anaerobic promoters are: P_{gapA}, P_{gadA}, P_{pflB}, Pₑₙₒ, P_{cysK};
further, the promoter P_{gapA} has a nucleotide sequence shown in SEQ ID NO. 1;
further, the promoter P_{gadA} has a nucleotide sequence shown in SEQ ID NO. 2;
further, the promoter P_{pflB} has a nucleotide sequence shown in SEQ ID NO. 3;
further, the promoter Pₑₙₒ has a nucleotide sequence shown in SEQ ID NO. 4;
further, the promoter P_{cysK} has a nucleotide sequence shown in SEQ ID NO. 5.

Another technical solution provided by the present disclosure is a recombinant Escherichia coli strain capable of efficiently synthesizing lactic acid monomers using sucrose. The recombinant strain is obtained through genetic recombination and genetic modification technology, which takes an Escherichia coli strain capable of efficiently synthesizing lactic acid monomers from the sugar metabolism intermediate pyruvate as the starting strain, and replaces the promoter of the sucrose metabolism operon cscAKB in the genome with an anaerobic promoter;
further, said anaerobic promoters include but are not limited to: P_{gapA}, P_{gadA}, P_{pflB}, Pₑₙₒ, P_{cysK}, P_{glnA}, P_{pgk}, P_{manX}, P_{adhE}, P_{pfkA}, P_{acnA}, P_{hchA}, P_{agp}, P_{serA}, P_{melA}, P_{tpiA}, P_{pykA}, P_{fruK}, P_{ackA}, P_{yqhD}, etc.;
preferably, said anaerobic promoters are: P_{gapA}, P_{gadA}, P_{pflB}, Pₑₙₒ, P_{cysK};
further, the starting strain adopted by the recombinant strain is Escherichia coli CGMCC No. 11059 or Escherichia coli CGMCC No. 11060.

The third technical solution provided by the present disclosure is the application of the recombinant strain according to the second technical solution, especially in the production of lactic acid monomers.

Further, it is the application in the synthesis of lactic acid monomers using sucrose as a raw material.

Further, the method for fermentative production of lactic acid monomers using the above recombinant strain is as follows:
Shake flask fermentation test: Inoculate the fermentation medium at an initial OD₆₀₀ value of 0.015~0.075, with an initial sucrose concentration of 0.5%~1% (w/v), ferment at 25° C.~37° C. with a pH of 6.5~7.5 and a shaker rotation speed of 100~250 r/min, culture until the OD₆₀₀ reaches 1.8~3.0, add sucrose with a final concentration of 1%~7% (w/v) and calcium carbonate with a final concentration of 1%~5% (w/v), then statically culture at 37° C.~50° C. for 12~24 hours;
Fermenter fermentation test: Inoculate the seed liquid into a fermenter containing fermentation medium at an initial OD₆₀₀ value of 0.1~1.0, with an initial sucrose concentration of 1%~7% (w/v), and carry out fermentation according to a two-stage fermentation method. In the cell growth stage, control the temperature at 25° C.~37° C., maintain the pH at 6.5~7.5, set the rotation speed at 1~1000 r/min, and control the dissolved oxygen at 20%~80%; when the cell concentration reaches an OD₆₀₀ of 10~50, enter the anaerobic lactic acid fermentation stage, control the temperature at 37° C.~50° C., adjust the stirring speed to 1~200 r/min, and separately fed-batch 30%~70% (w/v) sucrose solution and 5%~35% (w/v) calcium hydroxide suspension to maintain the sugar concentration in the fermentation broth at 0.5%~4% and the pH at 6.5~7.5; the total fermentation time is 24~36 hours.

The composition of the fermentation medium (in g/L) is: Na₂HPO₄·12H₂O 5~25, KH₂PO₄ 1~10, NH₄Cl 0.1~5, NaCl 0.2~1, MgSO₄ 0.01~0.5, and a trace element mother liquor 0.1~1.5 mL/L.

The composition of the trace element mother liquor (in g/L) is: FeCl₃·6H₂O 0.1~5, CoCl₂·6H₂O 0.1~0.5, CuCl₂·2H₂O 0.01~0.2, ZnCl₂ 0.1~0.5, Na₂MO₄·2H₂O 0.1~0.5, H₃BO₃ 0.01~0.1, MnCl₂·4H₂O 0.1~1.

### Beneficial Effects

The present disclosure creatively identifies anaerobic promoters that can efficiently initiate and guide the sucrose metabolism pathway. Applying this finding to strain improvement, the obtained new strain can metabolize sucrose as efficiently as glucose to synthesize and accumulate lactic acid monomers.

Using the recombinant strain of the present disclosure, the lactic acid production process can achieve efficient synthesis of lactic acid monomers using sucrose as a raw material without adding other raw materials (such as invertase). The present disclosure helps expand the raw material supply for large-scale production of lactic acid monomers, optimizes the production technology of lactic acid monomers from sucrose raw materials, and can be applied to the large-scale production of lactic acid monomers using sucrose as a raw material, further improving the economic benefits and industrial structure of sugarcane and sugar beet planting industries. The present disclosure can also be used for the development of efficient production of other biological products by Escherichia coli using sucrose as a raw material.

The recombinant strain of the present disclosure grows rapidly using sucrose at 25~37° C. to form cells; and rapidly synthesizes lactic acid monomers (D-lactic acid or L-lactic acid) with high optical purity and high chemical purity using sucrose at 37~50° C. It can directly and efficiently metabolize sucrose to synthesize lactic acid monomers, and has significant ability to catabolize sucrose for synthesizing high-purity lactic acid monomers. For example, the fermentation levels of D-lactic acid and L-lactic acid for strains DSAPW5 and LSAPW6 ferment at 25~50° C. for 28~30 hours can reach 158.58 g/L and 159.65 g/L, respectively, with optical purities of 99.94% and 99.95%.

### BRIEF DESCRIPTION OF FIGURES

FIG. 1: Selection of anaerobic promoters for sucrose metabolism, with the transcriptional strength of each promoter ranked from high to low.

FIG. 2: Flowchart of promoter sequence replacement in the sucrose metabolism gene cscAKB.

FIG. 3: PCR identification electrophoresis pattern of new sucrose metabolism strains DSAPW5 and LSAPW6,

Among them, Lane M: marker; Lane 1: PCR product of the wild-type strain; Lane 2 and Lane 3: PCR products of mutant strains DSAPW5 and LSAPW6, respectively.

### DETAILED DESCRIPTION

To make the purpose, technical solutions, and advantages of the present patent clearer, the present patent will be further described in detail below with reference to specific examples. It should be understood that the specific examples described herein are only used to explain the present patent and are not intended to limit the present disclosure.

The starting strains adopted in the present disclosure are metabolically engineered Escherichia coli CGMCC No. 11059 and Escherichia coli CGMCC No. 11060, which are D-lactic acid monomer-producing strain and L-lactic acid monomer-producing strain, respectively. They have been deposited in the China General Microbiological Culture Collection Center (CGMCC) under Budapest Treaty on Jul. 7, 2015.

The method for dynamic analysis of genome-wide transcription level adopted in the present disclosure: Using conventional laboratory lactic acid fermentation methods, sequencing samples were prepared in a 5 L fermenter with an initial fermentation medium volume of 2.5 L and a sucrose concentration of 30 g/L. Samples were taken at three fermentation stages: mid-logarithmic phase under aerobic conditions (cell density (OD600) reaching about 10), 3 hours of culture under anaerobic conditions, and recovery of aerobic growth (about 3 hours). Cells were collected by centrifugation at 5000×g for 5 minutes, quickly frozen in liquid nitrogen, and stored at −70° C. mRNA from samples of different fermentation stages was prepared by Guangzhou Genepioneer Biotechnologies Co., Ltd., and transcriptome analysis and sequencing were completed.

The main experimental methods adopted in the present disclosure are as follows:

### 1. Gene Cloning, Genetic Recombination, and Construction of Recombinant Plasmids

Conventional molecular cloning operations were performed with reference to literature methods (Sambrook. et al. Molecular Cloning: A Laboratory Manual, 1989).

### 2. Extraction of Chromosomal DNA

The method for extracting Escherichia coli chromosomal DNA was performed according to the literature (Jian Zhuge, Zhengxiang Wang. Experimental Technology Manual for Industrial Microbiology, China Light Industry Press, 1994).

### 3. Extraction of Plasmid DNA Plasmid

DNA was extracted using Sigma's plasmid mini-prep kit after lysing the cell wall with a certain concentration of lysozyme.

### 4. Gene Amplification

DNA amplification was performed in 0.2 mL PCR thin-walled tubes. The PCR amplification conditions were: 1×(95° C. for 5 min); 30×(94° C. for 10 s, 58° C. for 30 s, 72° C. for 30~300 s); 1×(72° C. for 10 min). Depending on the different amplification lengths, the extension temperature and time of the PCR reaction varied. Unless otherwise specified, all PCR reactions were performed using Pfu DNA polymerase.

5. Escherichia coli Gene Deletion and Site-Directed Integration

Refer to the method described in the literature (Li Zhou et al. Multiple Gene Inactivation Approach in Escherichia coli Mediated by a Combination of Red Recombination and Xer Recombination. Microbiology China, 2010, 37:923-928). The main steps are as follows: (1) Using Escherichia coli genomic DNA as a template, amplify the target gene sequence by PCR and clone it into a suitable vector, select appropriate restriction enzyme sites for digestion, clone the foreign gene expression cassette to be expressed into it; further clone the difEry fragment (coding sequence as listed) into it through the restriction enzyme sites existing outside the expression cassette, thereby obtaining the site-directed integration sequence with the foreign gene expression cassette (i.e., homology arm 1-expression cassette-dif-Ery-dif-homology arm 2). (2) Prepare the above site-directed integration expression cassette sequence by PCR amplification, purify the fragment, and electrotransform it into the recipient strain containing the helper plasmid pKD46. (3) Under the action of the Red recombinase produced by the helper plasmid pKD46, the above DNA fragment undergoes double crossover with the target gene on the chromosome, replacing the target gene and introducing the target gene expression cassette sequence. Recombinant transformants can be screened using the erythromycin resistance marker carried on the mutant cassette. The mutant strain then undergoes recombination at the two dif sites under the action of the Xer recombinase produced by itself to circularly remove the antibiotic resistance gene. Correct transformants were verified by colony PCR, plasmid extraction and digestion, fermentation verification of function, etc.

### 6. Evaluation of the Growth Status of Recombinant Strains

It was carried out in 250 mL Erlenmeyer flasks containing 50~100 mL fermentation medium. Inoculate at an initial OD₆₀₀ value of 0.015~0.075, with a sugar concentration of 0.1%~1.0%, ferment at 30° C.~37° C. with a pH of 6.5~7.5 and a shaker rotation speed of 100~250 r/min, culture for 12-24 hours, take samples regularly to determine the OD₆₀₀, and observe its growth status.

### 7. Lactic Acid Fermentation Test

The composition of the fermentation medium (in g/L) is: Na₂HPO₄·12H₂O 5~25, KH₂PO₄ 1~10, NH₄Cl 0.1~5, NaCl 0.2~1, MgSO₄ 0.01~0.5, and trace element mother liquor 0.1~1.5 mL/L.

The composition of the trace element mother liquor (in g/L) is: FeCl₃·6H₂O 0.1~5, CoCl₂·6H₂O 0.1~0.5, CuCl₂·2H₂O 0.01~0.2, ZnCl₂ 0.1~0.5, Na₂MO₄·2H₂O 0.1~0.5, H₃BO₃ 0.01~0.1, MnCl₂·4H₂O 0.1~1.

\textcircled{1} Shake flask fermentation test: The fermentation process was carried out in 250 mL Erlenmeyer flasks containing 50~100 mL fermentation medium. Inoculate at an initial OD₆₀₀ value of 0.015~0.075, with an initial sucrose concentration of 0.5%~1% (w/v), ferment at 25° C.~37° C. with a pH of 6.5~7.5 and a shaker rotation speed of 100~250 r/min, culture until the OD₆₀₀ reaches 1.8~3.0, add sucrose with a final concentration of 1%~7% (w/v) and calcium carbonate with a final concentration of 1%~5% (w/v), then statically culture at 37° C.~50° C. for 12~24 hours. Take samples regularly to determine the cell amount, residual sugar, and content of L-lactic acid or D-lactic acid in the fermentation broth.

\textcircled{2} Fermenter fermentation test: Pick a single colony of the fermentation strain, inoculate it into 50 mL LB liquid medium, and culture it in a shaker at 25° C.~37° C. and 100~250 r/min for 5~15 hours as the first-grade seed liquid. Inoculate the first-grade seed liquid into 100 mL fermentation medium supplemented with sucrose as the carbon source, with an initial sugar concentration of 0.1%~1.0%, and culture it in a shaker at 25° C.~37° C. and 100~250 r/min for 5~15 hours as the second-grade seed liquid. Inoculate the second-grade seed liquid into a fermenter containing fermentation medium at an initial OD₆₀₀ value of 0.1~1.0, with an initial sucrose concentration of 1~7% (w/v). The initial volume of the fermenter after inoculation is 25%~60% of the working volume, and fermentation is carried out according to a two-stage fermentation method. During the cell growth stage of the fermentation process, control the temperature at 25° C.~37° C., maintain the pH at 6.5~7.5, set the rotation speed at 1~1000 r/min, and control the dissolved oxygen at 20%~80%; when the cell concentration reaches an OD₆₀₀ of 10~50, enter the anaerobic lactic acid fermentation stage, control the temperature at 37° C.~50° C., adjust the stirring speed to 1~200 r/min, and separately fed-batch 30%~70% (w/v) sucrose solution and 5%~35% (w/v) calcium hydroxide suspension to maintain the sugar concentration in the fermentation broth at 0.5%~4% and the pH at 6.5~7.5. The total fermentation time is 24~36 hours. During the fermentation process, take samples regularly to analyze cell density, sugar consumption, lactic acid yield, main metabolic intermediates, and other organic acid products.

### 8. Analysis of Fermentation Broth Components:

Sample pretreatment: Take 1 mL fermentation broth, mix with 50 μL of 10 mol/L sulfuric acid, centrifuge at 12000 r/min for 5 minutes, aspirate an appropriate amount of supernatant, add an equal volume of absolute ethanol, mix well, let stand at 4° C. for 4 hours, centrifuge at 12000 r/min for 5 minutes. Dilute the supernatant appropriately with ddH₂O, filter through a 0.22 μm organic phase microporous membrane, and perform analysis and determination of related components.

\textcircled{1} Determination of glucose concentration: After appropriately diluting the sample, determine the glucose concentration using an SBA-40C biosensor, and take the average value of three parallel data.

\textcircled{2} Determination of sucrose concentration: Determined by HPLC method. Chromatographic analysis conditions: chromatographic column is Grace Prevail Carbohydrate ES 5u liquid chromatographic column, column temperature is 30° C., column pressure is 90 bar, mobile phase is 65% acetonitrile (v/v), flow rate is 1.0 mL/min, detector drift tube temperature is 90° C., air carrier gas flow rate is 2.2 mL/min, injection volume is 15 μL.

\textcircled{3} Determination of the content of organic acids such as D-lactic acid and L-lactic acid: Performed by HPLC. Chromatographic detection conditions: chromatographic column is HPX-87H organic acid analysis column, column temperature is 65° C., detection wavelength is 210 nm, mobile phase is 5 mmol/L sulfuric acid solution, flow rate is 0.6 mL/min, injection volume is 20 μL. All data are the average values of 3 parallel test results.

\textcircled{4} Determination of optical purity of lactic acid monomers: Performed by HPLC. Chromatographic detection conditions: chromatographic column is Astec CLC-L optical purity analysis column, column temperature is 25° C., detection wavelength is 254 nm, mobile phase is 5 mmol/L copper sulfate solution, flow rate is 1 mL/min, injection volume is 10 μL.

The present disclosure will be further explained and illustrated below through specific examples.

### Example 1 Preparation of Samples Based on Transcriptome Analysis of Sucrose Metabolism

Sucrose metabolism fermentation was carried out in a 5 L fermenter to prepare cell samples at different stages. Pick a single colony of the test strain CGMCC No. 11059 or CGMCC No. 11060, inoculate it into a 250 mL Erlenmeyer flask containing 50 mL LB liquid medium, and culture at 37° C. with a stirring rotation speed of 200 r/min until the OD₆₀₀ reaches 3.0 to prepare the first-grade seed liquid; inoculate the first-grade seed liquid into a 500 mL Erlenmeyer flask containing 100 mL fermentation medium with a sucrose concentration of 5 g/L, and culture at 37° C. with a stirring rotation speed of 200 r/min until the OD₆₀₀ reaches 3.0 to prepare the second-grade seed liquid; inoculate the second-grade seed liquid into a 5 L fermenter containing fermentation medium at an initial OD₆₀₀ value of 0.3, with a liquid volume of 2.5 L and an initial sucrose concentration of 30 g/L, and a fermentation temperature of 37° C.

The composition of the fermentation medium (in g/L) is: Na₂HPO₄·12H₂O 15.11, KH₂PO₄ 3, NH₄Cl 1, NaCl 0.5, MgSO₄ 0.12, and trace element mother liquor 1 mL/L.

The composition of the trace element mother liquor (in g/L) is: FeCl₃·6H₂O 2.4, CoCl₂·6H₂O 0.3, CuCl₂·2H₂O 0.15, ZnCl₂ 0.3, Na₂MO₄·2H₂O 0.3, H₃BO₃ 0.075, MnCl₂·4H₂O 0.495.

Sample preparation for transcriptome analysis was carried out by sampling at three stages. The first stage (S1) is the cell growth stage: maintain the pH at 7.0 with ammonia water, adjust the rotation speed and air flow rate to maintain the dissolved oxygen above 60%, aerobically culture the cells to the mid-logarithmic phase (OD₆₀₀ value of about 10), take 100 mL fermentation broth, centrifuge at 4° C. and 5000×g for 5 minutes, discard the supernatant to collect the cells, immediately freeze the collected cells in liquid nitrogen, and store at −70° C.; the second stage (S2) is the anaerobic fermentation stage: turn off the aeration, control the rotation speed at 200 r/min, maintain the pH at 7.0 with ammonia water, allow the cells to enter the anaerobic fermentation state (about 3 hours), take 100 mL fermentation broth, centrifuge at 4° C. and 5000×g for 5 minutes, discard the supernatant to collect the cells, immediately freeze the collected cells in liquid nitrogen, and store at −70° C.; the third stage (S3) is the recovery growth stage: resume aeration, adjust the rotation speed to maintain the dissolved oxygen above 60%, maintain the pH at 7.0 with 1 M hydrochloric acid, allow the cells to re-enter the aerobic growth state (about 3 hours), take 100 mL fermentation broth, centrifuge at 4° C. and 5000×g for 5 minutes, discard the supernatant to collect the cells, immediately freeze the collected cells in liquid nitrogen, and store at −70° C.

### Example 2 Selection of Promoters Capable of Mediating Sucrose Metabolism Under Anaerobic Conditions

Transcriptome samples were prepared according to the above fermentation method. mRNA of the strains at the three fermentation stages (S1-S3) was prepared by Guangzhou Genepioneer Biotechnologies Co., Ltd., and then sequenced on the Illumina sequencing platform. The present disclosure uses the whole-genome sequence of Escherichia coli W (NCBI database; CP002185.1) as the reference genome, uses TopHat2 to align the filtered Clean reads with the reference genome to obtain position information on the reference genome, and obtain the number of aligned reads and unaligned reads. Subsequent bioinformatics analysis was performed based on the number of aligned reads. Through the position information of the aligned reads on the reference genome, the FPKM method was used to quantify the expression levels of transcripts and genes in the samples. Differentially expressed genes were screened with a fold change (FC)≥2 and false discovery rate (FDR)<0.01, and classified into up-regulated genes and down-regulated genes according to relative expression levels. At the same time, functional annotation and enrichment analysis of differentially expressed genes were performed in various databases such as GO and KEGG.

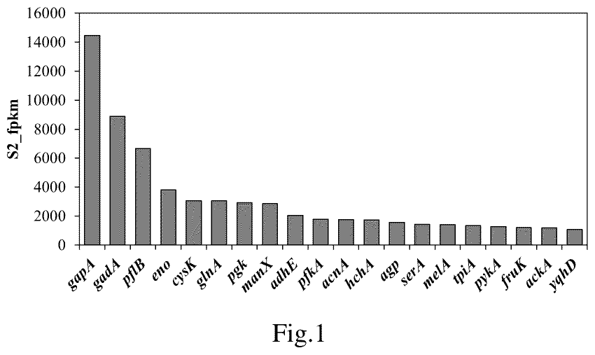

The present disclosure selects promoters according to the following criteria: 1) Promoters of genes involved in “carbohydrate metabolism” in the genome; 2) Promoters of genes whose expression level in the anaerobic fermentation stage (S2) is higher than that in the aerobic growth stage (S1); 3) The top 20 genes with relatively high expression levels. Based on the selection criteria that meet the above conditions, 20 corresponding promoters of genes were screened, namely P_{gapA}, P_{gadA}, P_{pflB}, Pₑₙₒ, P_{cysK}, P_{glnA}, P_{pgk}, P_{manX}, P_{adhE}, P_{pfkA}, P_{acnA}, P_{hchA}, P_{agp}, P_{serA}, P_{melA}, P_{tpiA}, P_{pykA}, P_{fruK}, P_{ackA}, P_{yqhD}. The comparison for expression intensity is shown in FIG. 1.

### Example 3 Construction of New Sucrose Metabolism Strains

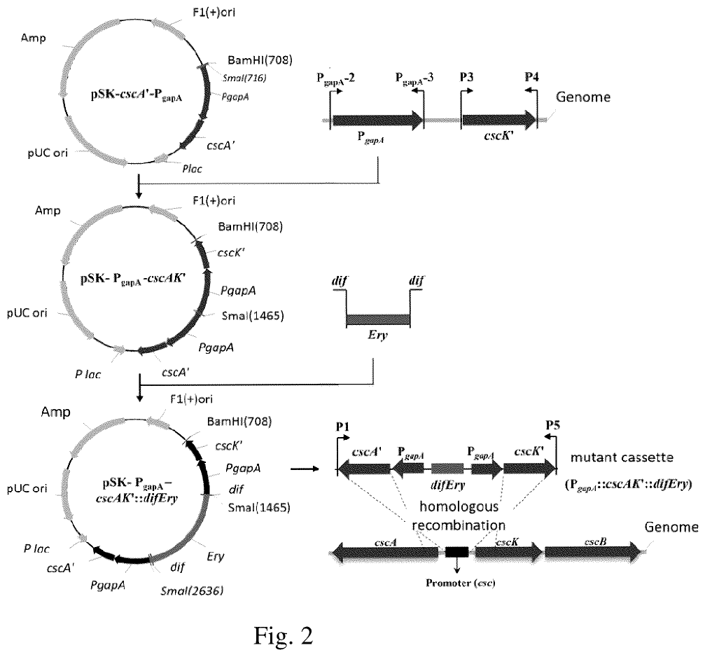

The top 20 promoters with the highest transcriptional strength mentioned above were selected as candidate promoters, namely P_{gapA}, P_{gadA}, P_{pflB}, Pₑₙₒ, P_{cysK}, P_{glnA}, P_{pgk}, P_{manX}, P_{adhE}, P_{pfkA}, P_{acnA}, P_{hchA}, P_{agp}, P_{serA}, P_{melA}, P_{tpiA}, P_{pykA}, P_{fruK}, P_{ackA}, P_{yqhD}. Based on the above gene deletion and integration method, the original promoter sequence of the sucrose metabolism operon cscAKB in the genome of Escherichia coli CGMCC No. 11059 or CGMCC No. 11060 was deleted and replaced with the candidate promoter. The experimental flow is shown in FIG. 2. Since the natural sucrose metabolism operon cscAKB has a bidirectional promoter between the cscA and cscK genes, which can simultaneously participate in the co-transcription of the cscKB gene and the reverse transcription of the cscA gene, in this example, the candidate promoter was reversely spliced by gene splicing to construct a promoter capable of bidirectionally regulating gene transcription. Taking the replacement of P_{gapA} as an example, the experimental steps are as follows.

Using the genome of Escherichia coli CGMCC No. 11059 or CGMCC No. 11060 as a template, amplify the homologous sequence upstream of the original promoter in the sucrose metabolism operon cscAKB (i.e., a partial fragment of the cscA gene) by PCR with primer P1 and primer P2, and simultaneously amplify the sequence P_{gapA} for initiating cscA gene transcription by PCR with primers P_{gapA}-1 and P_{gapA}-2. Then, overlap PCR was performed with primer P1 and P_{gapA}-2 to obtain the gene sequence cscA′-P_{gapA} with the upstream homologous sequence and the corresponding promoter, and this fragment was cloned into the Smal restriction enzyme site of plasmid pSKsym (published in Overhage J. et al. Biotransformation of eugenol to vanillin by a mutant of Pseudomonas sp. strain HR199 constructed by disruption of the vanillin dehydrogenase (vdh) gene. Appl Microbiol Biotechnol, 1999, 52, 820-828) to obtain the recombinant plasmid pSK-cscA′-P_{gapA}.

Using the genome of Escherichia coli CGMCC No. 11059 or CGMCC No. 11060 as a template, amplify the sequence P_{gapA} for initiating the transcription of cscK and cscB genes by PCR with primers P_{gapA}-2 and P_{gapA}-3, and simultaneously amplify the homologous sequence downstream of the original promoter in the sucrose metabolism operon cscAKB (i.e., a partial fragment of the cscK gene) by PCR with primers P3 and P4. Then, overlap PCR was performed with primers P_{gapA}-3 and P4 to obtain the gene sequence P_{gapA}-cscK′ with the downstream homologous sequence and the corresponding promoter. This fragment and plasmid pSK-cscA′-P_{gapA} were double-digested with restriction enzymes Smal and BamHI, and ligated to obtain the recombinant plasmid pSK-P_{gapA}-cscAK′, which contains two promoters P gapA in the plasmid, capable of simultaneously initiating the transcription of cscA and cscKB genes.

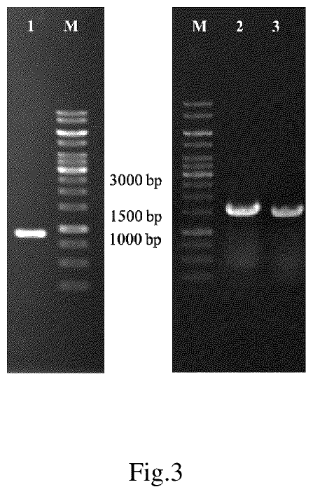

The erythromycin resistance gene fragment difEry with dif sequence (shown in SEQ ID NO. 6) was cloned into the Smal restriction enzyme cutting site of the above recombinant plasmid pSK-P_{gapA}-cscAK′ to obtain the recombinant plasmid pSK-P_{gapA}-cscAK′::difEry. Using this recombinant plasmid as a template, PCR amplification was performed with primers P1 and P5 to obtain the mutant cassette P_{gapA}-cscAK′::difEry. This fragment was transformed into Escherichia coli CGMCC No. 11059 and CGMCC No. 11060, transformants were screened on LB medium with erythromycin resistance (160 μg/mL), and then passaged on non-selective LB medium to screen out transformants with loss of erythromycin resistance. The chromosomal DNA of the transformants was extracted and verified by PCR (using verification primers P1 and P5 in Table 1; the band size of the wild-type strain is about 800 bp, and the band size of the mutant strain is about 1500 bp; the verification result is shown in FIG. 3). Mutant strains Escherichia coli DSAPW5 and LSAPW6 were obtained.

Using the above similar method to replace the other 19 promoters, the expression cassettes P_{gadA}-cscAK′::difEry, P_{pflB}-cscAK′::difEry, Pₑₙₒ-cscAK′::difEry, P_{cysK}-cscAK′::difEry, P_{glnA}-cscAK′::difEry, P_{pgk}-cscAK′::difEry, P_{manX}-cscAK′::difEry, P_{adhE}-cscAK′::difEry, P_{pfkA}-cscAK′::difEry, P_{acnA}-cscAK′::difEry, P_{hchA}-cscAK′::difEry, P_{agp}-cscAK′::difEry, P_{serA}-cscAK′::difEry, P_{melA}-cscAK′::difEry, P_{tpiA}-cscAK′::difEry, P_{pykA}-cscAK′::difEry, P_{fruK}-cscAK′: difEry, P_{ackA}-cscAK′::difEry, and P_{yqhD}-cscAK′::difEry were prepared by replacing the corresponding primers (as shown in Table 1). The expression cassettes were respectively transformed into Escherichia coli CGMCC No. 11059 and CGMCC No. 11060 to obtain mutant strains DSADW5/LSADW6, DSPLW5/LSPLW6, DSENW5/LSENW6, DSYSW5/LSYSW6, DSLNW5/LSLNW6, DSPGW5/LSPGW6, DSANW5/LSANW6, DSDHW5/LSDHW6, DSFKW5/LSFKW6, DSCNW5/LSCNW6, DSCHW5/LSCHW5, DSAGW5/LSAGW6, DSERW5/LSERW6, DSELW5/LSELW6, DSPIW5/LSPIW6, DSYKW5/LSYKW6, DSRUW5/LSRUW6, DSCKW5/LSCKW6, and DSQHW5/LSQHW6, as shown in Table 2.

TABLE 1
List of Primers Used in This Case

| Nama | | Restriction | SEQ |
|---|---|---|---|
| of | | Enzyme | ID |
| primers | nucleotide sequence (5′->3′) | cutting site | No. |
| P1 | CCTGAAAAACACCCGTCTTTG | BamHI | 7 |
| P2 | ATGACGCAATCTCGATTGCA | | 8 |
| P3 | ATGTCAGCCAAAGTATGGGT | | 9 |
| P4 | ATTGGATCCATAAACGTAAATGAACGTTC | | 10 |
| P5 | ATAAACGTAAATGAACGTTC | | 11 |
| P_{gapA}-1 | TCGAGATTGCGTCATATATTCCACCAGCTATTTGT | SmaI/BamHI | 12 |
| P_{gapA}-2 | ATTGGATCCCCCGGGGCAGTAAACGACCCGTAAAT | | 13 |
| P_{gapA}-3 | CCATACTTTGGCTGACATATATTCCACCAGCTATTTGT | | 14 |
| P_{gadA}-1 | TCGAGATTGCGTCATTTCGAACTCCTTAAATTTAT | SmaI/BamHI | 15 |
| P_{gadA}-2 | ATTGGATCCCCCGGGTTAATTTGATCGCCCGAACA | | 16 |
| P_{gadA}-3 | CCATACTTTGGCTGACATTTCGAACTCCTTAAATTTAT | | 17 |
| P_{pflB}-1 | TCGAGATTGCGTCATGTAACACCTACCTTCTTAAG | SmaI/BamHI | 18 |
| P_{pflB}-2 | ATTGGATCCCCCGGGTGAGTTATTCTGGCCGCAGCC | | 19 |
| P_{pflB}-3 | CCATACTTTGGCTGACATGTAACACCTACCTTCTTAAG | | 20 |
| Pₑₙₒ-1 | TCGAGATTGCGTCATTTCCTCAAGTCACTAGTTAA | SmaI/BamHI | 21 |
| Pₑₙₒ-2 | ATTGGATCCCCCGGGTTGCCAGTTCCATCCGGAGT | | 22 |
| Pₑₙₒ-3 | CCATACTTTGGCTGACATTTCCTCAAGTCACTAGTTAA | | 23 |
| P_{cysK}-1 | TCGAGATTGCGTCATGGCCTGTCCTTAACTGTATG | SmaI/BamHI | 24 |
| P_{cysK}-2 | ATTGGATCCCCCGGGCAATCTACCGGTTATTTTGT | | 25 |
| P_{cysK}-3 | CCATACTTTGGCTGACATGGCCTGTCCTTAACTGTATG | | 26 |
| P_{glnA}-1 | TCGAGATTGCGTCATACTTTAACTCTCCTGGATTGGTC | SmaI/BamHI | 27 |
| P_{glnA}-2 | ATTGGATCCCCCGGGAACTTTGCCTCAGGCATTAG | | 28 |
| P_{glnA}-3 | CCATACTTTGGCTGACATACTTTAACTCTCCTGGATTGGTC | | 29 |
| P_{pgk}-1 | TCGAGATTGCGTCATGGTGAATCCTCTCGTTGATTCT | SmaI/BamHI | 30 |
| P_{pgk}-2 | ATTGGATCCCCCGGGTACGGAATTGCCGTTGGTCT | | 31 |
| P_{pgk}-3 | CCATACTTTGGCTGACATGGTGAATCCTCTCGTTGATTCT | | 32 |
| P_{manX}-1 | TCGAGATTGCGTCATTTGCTACCTCCTTTATTATCGT | SmaI/BamHI | 33 |
| P_{manX}-2 | ATTGGATCCCCCGGGACTGCGGGCTACTGCCCTAT | | 34 |
| P_{manX}-3 | CCATACTTTGGCTGACATTTGCTACCTCCTTTATTATCGT | | 35 |
| P_{adhE}-1 | TCGAGATTGCGTCATAATGCTCTCCTGATAATGTTA | SmaI/BamHI | 36 |
| P_{adhE}-2 | ATTGGATCCCCCGGGTTTTCACCTCCTAACTACTTA | | 37 |
| P_{adhE}-3 | CCATACTTTGGCTGACATAATGCTCTCCTGATAATGTTA | | 38 |
| P_{pfkA}-1 | TCGAGATTGCGTCATGACTACCTCTGAACTTTGGA | SmaI/BamHI | 39 |
| P_{pfkA}-2 | ATTGGATCCCCCGGGTCAGTATAAAAGAGAGCCAG | | 40 |
| P_{pfkA}-3 | CCATACTTTGGCTGACATGACTACCTCTGAACTTTGGA | | 41 |
| P_{acnA}-1 | TCGAGATTGCGTCATAGCTCCTCCTTAATGACAGG | SmaI/BamHI | 42 |
| P_{acnA}-2 | ATTGGATCCCCCGGGAACTGTTTGCTGAAGATGAT | | 43 |
| P_{acnA}-3 | CCATACTTTGGCTGACATAGCTCCTCCTTAATGACAGG | | 44 |
| P_{hchA}-1 | TCGAGATTGCGTCATAGTGTATTCCTTATTGTTGCTT | SmaI/BamHI | 45 |
| P_{hchA}-2 | ATTGGATCCCCCGGGAAGCCACTTGCGACGACGTT | | 46 |
| P_{hchA}-3 | CCATACTTTGGCTGACATAGTGTATTCCTTATTGTTGCTT | | 47 |
| P_{agp}-1 | TCGAGATTGCGTCATTCCTGGCACCTCTTTTGTTA | SmaI/BamHI | 48 |
| P_{agp}-2 | ATTGGATCCCCCGGGTGGCAAAGGCATATGCTAAA | | 49 |
| P_{agp}-3 | CCATACTTTGGCTGACATTCCTGGCACCTCTTTTGTTA | | 50 |
| P_{serA}-1 | TCGAGATTGCGTCATTTACCCAATCCTGTCTTTTG | SmaI/BamHI | 51 |
| P_{serA}-2 | ATTGGATCCCCCGGGTCTGACGGGGGAACCTCCC | | 52 |
| P_{serA}-3 | CCATACTTTGGCTGACATTTACCCAATCCTGTCTTTTG | | 53 |
| P_{melA}-1 | TCGAGATTGCGTCATGCAGATCTCCTGGCTTGCTT | SmaI/BamHI | 54 |
| P_{melA}-2 | ATTGGATCCCCCGGGGGATGGCTCTCTTTCCTGGA | | 55 |
| P_{melA}-3 | CCATACTTTGGCTGACATGCAGATCTCCTGGCTTGCTT | | 56 |
| P_{tpiA}-1 | TCGAGATTGCGTCATTTTAATTCTCCACGCTTATAAG | SmaI/BamHI | 57 |
| P_{tpiA}-2 | ATTGGATCCCCCGGGCAAAAAGCAAAGCCTTTGTG | | 58 |
| P_{tpiA}-3 | CCATACTTTGGCTGACATTTTAATTCTCCACGCTTATAAG | | 59 |
| P_{pykA}-1 | TCGAGATTGCGTCATGTAATACTCCGTTGACTGAA | SmaI/BamHI | 60 |
| P_{pykA}-2 | ATTGGATCCCCCGGGGTTACTTAATTTAAGTGACG | | 61 |
| P_{pykA}-3 | CCATACTTTGGCTGACATGTAATACTCCGTTGACTGAA | | 62 |
| P_{fruk}-1 | TCGAGATTGCGTCATAGTTCTCCTCTCTTGCTGAA | SmaI/BamHI | 63 |
| P_{fruk}-2 | ATTGGATCCCCCGGGGCTGGTCGATCACTGGAA | | 64 |
| P_{fruk-}3 | CCATACTTTGGCTGACATAGTTCTCCTCTCTTGCTGAA | | 65 |
| P_{ackA}-1 | TCGAGATTGCGTCATGGAAGTACCTATAATTGATACG | SmaI/BamHI | 66 |
| P_{ackA}-2 | ATTGGATCCCCCGGGGCTTCACCTCAACTTCACAT | | 67 |
| P_{ackA}-3 | CCATACTTTGGCTGACATGGAAGTACCTATAATTGATACG | | 68 |
| P_{yqhD}-1 | TCGAGATTGCGTCATTACTTGCTCCCTTTGCTGG | SmaI/BamHI | 69 |
| P_{yqhD}-2 | ATTGGATCCCCCGGGTTCTCCAGCACTCTGGAGAA | | 70 |
| P_{yqhD}-3 | CCATACTTTGGCTGACATTACTTGCTCCCTTTGCTGG | | 71 |

TABLE 2
Genetic Characteristics of New Sucrose Metabolism Strains

| Strain No. | Strain | Strain Characteristics |
|---|---|---|
| 1 | CGMCC 11059 | D-lactic acid-producing strain |
| 2 | CGMCC 11060 | L-lactic acid-producing strain |
| 3 | DSAPW5 | CGMCC 11059::PgapA |
| 4 | DSADW5 | CGMCC 11059::PgadA |
| 5 | DSPLW5 | CGMCC 11059::PpflB |
| 6 | DSENW5 | CGMCC 11059::Peno |
| 7 | DSYSW5 | CGMCC 11059::PcysK |
| 8 | DSLNW5 | CGMCC 11059::PglnA |
| 9 | DSPGW5 | CGMCC 11059::Ppgk |
| 10 | DSANW5 | CGMCC 11059::PmanX |
| 11 | DSDHW5 | CGMCC 11059::PadhE |
| 12 | DSFKW5 | CGMCC 11059::PpfkA |
| 13 | DSCNW5 | CGMCC 11059::PacnA |
| 14 | DSCHW5 | CGMCC 11059::PhchA |
| 15 | DSAGW5 | CGMCC 11059::Pagp |
| 16 | DSERW5 | CGMCC 11059::PserA |
| 17 | DSELW5 | CGMCC 11059::PmelA |
| 18 | DSPIW5 | CGMCC 11059::PtpiA |
| 19 | DSYKW5 | CGMCC 11059::PpykA |
| 20 | DSRUW5 | CGMCC 11059::PfruK |
| 21 | DSCKW5 | CGMCC 11059::PackA |
| 22 | DSQHW5 | CGMCC 11059::PyqhD |
| 23 | LSAPW6 | CGMCC 11060::PgapA |
| 24 | LSADW6 | CGMCC 11060::PgadA |
| 25 | LSPLW6 | CGMCC 11060::PpflB |
| 26 | LSENW6 | CGMCC 11060::Peno |
| 27 | LSYSW6 | CGMCC 11060::PcysK |
| 28 | LSLNW6 | CGMCC 11060::PglnA |
| 29 | LSPGW6 | CGMCC 11060::Ppgk |
| 30 | LSANW6 | CGMCC 11060::PmanX |
| 31 | LSDHW6 | CGMCC 11060::PadhE |
| 32 | LSFKW6 | CGMCC 11060::PpfkA |
| 33 | LSCNW6 | CGMCC 11060::PacnA |
| 34 | LSCHW6 | CGMCC 11060::PhchA |
| 35 | LSAGW6 | CGMCC 11060::Pagp |
| 36 | LSERW6 | CGMCC 11060::PserA |
| 37 | LSELW6 | CGMCC 11060::PmelA |
| 38 | LSPIW6 | CGMCC 11060::PtpiA |
| 39 | LSYKW6 | CGMCC 11060::PpykA |
| 40 | LSRUW6 | CGMCC 11060::PfruK |
| 41 | LSCKW6 | CGMCC 11060::PackA |
| 42 | LSQHW6 | CGMCC 11060::PyqhD |

### Example 4 Fermentative Production of Lactic Acid Monomers by Recombinant Strains Using Sucrose as Raw Material

The recombinant strains in Table 2 were used as fermentation strains to evaluate their ability to produce lactic acid monomers from sucrose in a 5 L fermenter.

Pick a single colony of the fermentation strain, inoculate it into a 250 mL Erlenmeyer flask containing 50 mL LB liquid medium, and culture at 37° C. with a rotation speed of 200 r/min until the OD₆₀₀ reaches 3.0 to prepare the first-grade seed liquid; inoculate the first-grade seed liquid into a 500 mL Erlenmeyer flask containing 100 mL fermentation medium with a sucrose concentration of 5 g/L, and culture at 37° C. with a rotation speed of 200 r/min until the OD₆₀₀ reaches 3.0 to prepare the second-grade seed liquid. Inoculate the second-grade seed liquid into a 5 L fermenter containing 2.5 L fermentation medium at an initial OD₆₀₀ value of about 0.3, with an initial sucrose concentration of 3%, a fermentation temperature of 37° C., adjust the air flow rate and rotation speed to control the dissolved oxygen not less than 60%, maintain the pH at 7.0 with ammonia water, carry out aerobic culture until the OD₆₀₀ reaches about 30, set the fermenter temperature to 40° C., turn off the aeration, set the stirring rotation speed to 200 r/min, enter the anaerobic fermentation stage, fed-batch 60% sucrose solution to maintain the sugar concentration in the fermentation broth at 1%, fed-batch 25% calcium hydroxide suspension to maintain the pH at 7.0, and end the fermentation after determining that the lactic acid content no longer increases and the sucrose concentration is lower than 0.1%. The total fermentation time is 30 hours.

The composition of the fermentation medium (in g/L) is: Na₂HPO₄·12H₂O 15.11, KH₂PO₄ 3, NH₄Cl 1, NaCl 0.5, MgSO₄ 0.12, and trace element mother liquor 1 mL/L.

The composition of the trace element mother liquor (in g/L) is: FeCl₃·6H₂O 2.4, CoCl₂·6H₂O 0.3, CuCl₂·2H₂O 0.15, ZnCl₂ 0.3, Na₂MO₄·2H₂O 0.3, H₃BO₃ 0.075, MnCl₂·4H₂O 0.495.

Using the above fermentation technology, at the end of fermentation, the accumulation concentration, sugar-acid conversion rate, optical purity, and chemical purity of D-lactic acid and L-lactic acid of the strains replaced with anaerobic promoters are shown in Table 3.

TABLE 3
Production of Lactic Acid Monomers
Using Sucrose as Raw Material

| | | Final | Sugar-Acid | Optical | Chemical |
|---|---|---|---|---|---|
| | | Concentration | Conversion | Purity | Purity |
| No. | Strain | (g/L) | Rate (%) | (%) | (%) |
| 1 | CGMCC | 42.61 | 97.1 | 99.81 | 98.1 |
| | 11059 | | | | |
| 2 | CGMCC | 41.93 | 94.6 | 99.54 | 98.2 |
| | 11060 | | | | |
| 3 | DSAPW5 | 158.58 | 102.3 | 99.97 | 98.3 |
| 4 | DSADW5 | 149.16 | 101.0 | 99.94 | 98.3 |
| 5 | DSPLW5 | 144.57 | 101.8 | 99.96 | 98.4 |
| 6 | DSENW5 | 154.04 | 101.1 | 99.93 | 98.5 |
| 7 | DSYSW5 | 155.72 | 101.3 | 99.94 | 98.5 |
| 8 | DSLNW5 | 134.20 | 101.1 | 99.95 | 98.2 |
| 9 | DSPGW5 | 109.95 | 100.6 | 99.96 | 98.0 |
| 10 | DSANW5 | 138.77 | 101.5 | 99.94 | 98.0 |
| 11 | DSDHW5 | 94.04 | 98.8 | 99.93 | 98.2 |
| 12 | DSFKW5 | 101.46 | 98.1 | 99.93 | 98.1 |
| 13 | DSCNW5 | 42.43 | 94.6 | 99.95 | 98.3 |
| 14 | DSCHW5 | 69.97 | 95.0 | 99.94 | 98.0 |
| 15 | DSAGW5 | 54.57 | 96.5 | 99.95 | 98.2 |
| 16 | DSERW5 | 57.04 | 97.7 | 99.94 | 98.2 |
| 17 | DSELW5 | 45.70 | 93.7 | 99.94 | 98.1 |
| 18 | DSPIW5 | 55.50 | 94.0 | 99.93 | 97.8 |
| 19 | DSYKW5 | 42.27 | 93.2 | 99.94 | 98.2 |
| 20 | DSRUW5 | 44.81 | 93.9 | 99.95 | 98.3 |
| 21 | DSCKW5 | 41.04 | 95.8 | 99.93 | 98.3 |
| 22 | DSQHW5 | 43.54 | 94.6 | 99.93 | 97.9 |
| 23 | LSAPW6 | 159.65 | 102.1 | 99.95 | 98.6 |
| 24 | LSADW6 | 148.05 | 101.6 | 99.95 | 98.6 |
| 25 | LSPLW6 | 143.17 | 101.5 | 99.96 | 98.5 |
| 26 | LSENW6 | 152.33 | 101.6 | 99.95 | 98.5 |
| 27 | LSYSW6 | 156.20 | 101.8 | 99.97 | 98.4 |
| 28 | LSLNW6 | 136.91 | 101.3 | 99.93 | 98.3 |
| 29 | LSPGW6 | 111.54 | 99.5 | 99.96 | 98.3 |
| 30 | LSANW6 | 128.77 | 101.1 | 99.95 | 98.4 |
| 31 | LSDHW6 | 102.60 | 99.0 | 99.94 | 98.5 |
| 32 | LSFKW6 | 97.15 | 98.0 | 99.93 | 98.5 |
| 33 | LSCNW6 | 55.80 | 94.2 | 99.94 | 98.3 |
| 34 | LSCHW6 | 62.17 | 94.9 | 99.93 | 98.3 |
| 35 | LSAGW6 | 57.45 | 96.1 | 99.95 | 98.4 |
| 36 | LSERW6 | 50.04 | 94.9 | 99.93 | 98.5 |
| 37 | LSELW6 | 44.72 | 93.6 | 99.93 | 98.5 |
| 38 | LSPIW6 | 49.10 | 93.6 | 99.93 | 98.3 |
| 39 | LSYKW6 | 53.90 | 95.7 | 99.95 | 98.3 |
| 40 | LSRUW6 | 41.48 | 93.9 | 99.93 | 97.4 |
| 41 | LSCKW6 | 40.53 | 95.4 | 99.93 | 98.5 |
| 42 | LSQHW6 | 42.33 | 94.8 | 99.95 | 98.5 |

As can be seen from the results in Table 3, there are significant differences in the effects of the 20 promoter replacements, with the yield ranging from 100% to 380% of that of the original strain. It is evident that not all anaerobic promoter replacements can improve lactic acid yield. Among them, replacing the original promoter of cscAKB with P_{gapA}, P_{gadA}, P_{pflB}, Pₑₙₒ, or P_{cysK} results in a significant improvement in lactic acid yield, achieving unexpected technical effects.

### Example 5 Efficient Production of Lactic Acid Monomers by Recombinant Strains Fermenting Sucrose Under Different Fermentation Processes

The seed liquids of DSAPW5, DSYSW5, LSAPW6, and LSYSW6 were respectively inoculated into 5 L fermenters containing 2.5 L fermentation medium at an inoculum size corresponding to an initial OD₆₀₀ value of 0.1, with an initial sucrose concentration of 2.5% (w/v). Fermentation was carried out according to a two-stage fermentation method. In the cell growth stage, the temperature was controlled at 35° C., the pH was maintained at 7.0, the rotation speed was 200~800 r/min, and the dissolved oxygen was controlled at 40%; when the cell concentration reached an OD₆₀₀ of 25, the anaerobic lactic acid fermentation stage was initiated, with the temperature controlled at 40° C., the stirring rotation speed adjusted to 100 r/min, and 40% (w/v) sucrose solution and 20% (w/v) calcium hydroxide suspension fed-batch separately to maintain the sugar concentration in the fermentation broth at 2% and the pH at 7.0; the total fermentation time was 32 hours.

The composition of the fermentation medium (in g/L) was: Na₂HPO₄·12H₂O 15.11, KH₂PO₄ 3, NH₄Cl 1, NaCl 0.5, MgSO₄ 0.12, and trace element mother liquor 1 mL/L.

The composition of the trace element mother liquor (in g/L) was: FeCl₃·6H₂O 2.4, CoCl₂·6H₂O 0.3, CuCl₂·2H₂O 0.15, ZnCl₂ 0.3, Na₂MO₄·2H₂O 0.3, H₃BO₃ 0.075, MnCl₂·4H₂O 0.495.

The fermentation results are shown in Table 4 below.

TABLE 4

| | Lactic | Sugar-to- | | | |
|---|---|---|---|---|---|
| | Acid | Lactic Acid | Production | Optical | Chemical |
| | Yield | Conversion | Intensity[g/ | Purity | Purity |
| Strain | (g/L) | Rate (%) | (L · h)] | (%) | (%) |
| DSAPW5 | 147.64 | 101.7 | 4.61 | 99.95 | 98.5 |
| DSYSW5 | 150.13 | 101.3 | 4.69 | 99.95 | 98.4 |
| LSAPW6 | 148.65 | 102.1 | 4.65 | 99.93 | 98.6 |
| LSYSW6 | 153.20 | 101.5 | 4.79 | 99.95 | 98.3 |

### Example 6 Efficient Production of Lactic Acid Monomers by Recombinant Strains Fermenting Sucrose Under Different Fermentation Processes

The seed liquids of DSAPW5, DSYSW5, LSAPW6, and LSYSW6 were respectively inoculated into 5 L fermenters containing 2.5 L fermentation medium at an inoculum size corresponding to an initial OD₆₀₀ value of 0.5, with an initial sucrose concentration of 4% (w/v). Fermentation was conducted according to a two-stage fermentation method. In the cell growth stage, the temperature was controlled at 37° C., the pH was maintained at 6.8, the rotation speed was 200~800 r/min, and the dissolved oxygen was controlled at 50%; when the cell concentration reached an OD₆₀₀ of 30, the anaerobic lactic acid fermentation stage was started, with the temperature controlled at 42° C., the stirring rotation speed adjusted to 150 r/min, and 60% (w/v) sucrose solution and 25% (w/v) calcium hydroxide suspension fed-batch separately to maintain the sugar concentration in the fermentation broth at 0.5% and the pH at 6.8; the total fermentation time was 28 hours.

The composition of the fermentation medium (in g/L) was: Na₂HPO₄·12H₂O 15.11, KH₂PO₄ 3, NH₄Cl 1, NaCl 0.5, MgSO₄ 0.12, and trace element mother liquor 1 mL/L; The composition of the trace element mother liquor (in g/L) was: FeCl₃·6H₂O 2.4, CoCl₂·6H₂O 0.3, CuCl₂·2H₂O 0.15, ZnCl₂ 0.3, Na₂MO₄·2H₂O 0.3, H₃BO₃ 0.075, MnCl₂·4H₂O 0.495.

The fermentation results are shown in Table 5 below.

TABLE 5

| | Lactic | Sugar-to- | | | |
|---|---|---|---|---|---|
| | Acid | Lactic Acid | Production | Optical | Chemical |
| | Yield | Conversion | Intensity[g/ | Purity | Purity |
| Strain | (g/L) | Rate (%) | (L · h)] | (%) | (%) |
| DSAPW5 | 159.33 | 102.1 | 5.69 | 99.97 | 98.6 |
| DSYSW5 | 157.91 | 102.2 | 5.64 | 99.97 | 98.5 |
| LSAPW6 | 157.45 | 102.2 | 5.62 | 99.95 | 98.6 |
| LSYSW6 | 158.60 | 101.9 | 5.66 | 99.95 | 98.6 |

Although the present invention has been disclosed above with preferred embodiments, it is not intended to limit the present invention. Any person skilled in the art may make various changes, modifications, substitutions, and variations in form and details to these embodiments without departing from the spirit and principle of the present invention. The scope of the present invention is defined by the claims and their equivalents.

1. A method for improving the ability of Escherichia coli to synthesize lactic acid monomers using sucrose, wherein, the method is replacing a promoter of the sucrose metabolism operon cscAKB in the genome of the lactic acid monomer-producing strain with an anaerobic promoter;
wherein the anaerobic promoter is selected from: P_{gapA}, P_{gadA}, P_{pflB}, Pₑₙₒ, or P_{cysK};
the lactic acid monomer-producing strain is Escherichia coli CGMCC No. 11059 or Escherichia coli CGMCC No. 11060;
the nucleotide sequence of the promoter P_{gapA} is as shown in SEQ ID NO. 1; the nucleotide sequence of the promoter P_{gadA} is as shown in SEQ ID NO. 2; the nucleotide sequence of the promoter P_{pflB} is as shown in SEQ ID NO. 3; the nucleotide sequence of the promoter Pₑₙₒ is as shown in SEQ ID NO. 4; the nucleotide sequence of the promoter P_{cysK} is as shown in SEQ ID NO. 5.

2. An Escherichia coli recombinant strain capable of synthesizing lactic acid monomers using sucrose, wherein, the recombinant strain is obtained by taking an Escherichia coli strain capable of synthesizing lactic acid monomers from the sugar metabolism intermediate pyruvate as the starting strain, and replacing the promoter of the sucrose metabolism operon cscAKB in the genome with an anaerobic promoter;
wherein, the anaerobic promoter is selected from: P_{gapA}, P_{gadA}, P_{pflB}, Pₑₙₒ, or P_{cysK};
the starting strain adopted by the recombinant strain is Escherichia coli CGMCC No. 11059 or Escherichia coli CGMCC No. 11060;
the nucleotide sequence of the promoter P_{gapA} is as shown in SEQ ID NO. 1; the nucleotide sequence of the promoter P_{gadA} is as shown in SEQ ID NO. 2; the nucleotide sequence of the promoter P_{pflB} is as shown in SEQ ID NO. 3; the nucleotide sequence of the promoter Pₑₙₒ is as shown in SEQ ID NO. 4; the nucleotide sequence of the promoter P_{cysK} is as shown in SEQ ID NO. 5.

3. Application of the recombinant strain according to claim 2 in the production of lactic acid monomers.

4. The application according to claim 3, wherein, the application is in the production of lactic acid monomers using sucrose as a raw material.

5. The application according to claim 3, wherein, a shake flask fermentation method is as follows: inoculating the production strain into the fermentation medium at an initial OD₆₀₀ value of 0.015~0.075, with an initial sucrose concentration of 0.5%~1%, culturing at 25° C.~37° C. until the OD₆₀₀ reaches 1.8~3.0, adding sucrose with a final concentration of 1%~7% and calcium carbonate with a final concentration of 1%~5%, then statically culturing at 37° C.~50° C. for 12~24 hours.

6. The application according to claim 3, wherein, a fermenter fermentation method is as follows: inoculating the seed liquid into the fermentation medium at an initial OD₆₀₀ value of 0.1~1.0, with an initial sucrose concentration of 1%~7%, and the fermentation is carried out according to a two-stage fermentation method, a cell growth stage, controlling the temperature at 25° C.~37° C. and the dissolved oxygen at 20%~80%; when the cell concentration reaches an OD₆₀₀ of 10~50, entering into an anaerobic lactic acid fermentation stage, controlling the temperature at 37° C.~50° C., and separately feeding sucrose solution and calcium hydroxide suspension in a fed-batch manner to maintain the sugar concentration in the fermentation broth at 0.5%~4% and the pH at 6.5~7.5; the total fermentation time is 24~36 hours.

7. The application according to claim 6, wherein, the composition of the fermentation medium (in g/L) is: Na₂HPO₄·12H₂O 5~25, KH₂PO₄ 1~10, NH₄Cl 0.1~5, NaCl 0.2~1, MgSO₄ 0.01~0.5, and trace element mother liquor 0.1~1.5 mL/L.

8. The application according to claim 7, wherein, the composition of the trace element mother liquor (in g/L) is: FeCl₃·6H₂O 0.1~5, CoCl₂·6H₂O 0.1~0.5, CuCl₂·2H₂O 0.01~0.2, ZnCl₂ 0.1~0.5, Na₂MO₄·2H₂O 0.1~0.5, H₃BO₃ 0.01~0.1, MnCl₂·4H₂O 0.1~1.


SEQUENCE LISTING (71 sequences)

SEQ ID NO: 1 - DNA, 450 bp, Escherichia coli
  source 1..450: mol_type = genomic DNA
gcagtaaacgacccgtaaatggcggctctgtcccatgattctgcgtcacgtaaaactgcatctcggacaaattttttttcagttcttctgccgaaggtttattagccatttgctcacatctcactttaatcgtgctcacattacgtgactgattctaacaaaacattaacaccaactggcaaaattttgtcctaaacttgatctcgacgaaatggctgcacctaaatcgtgatgaaaatcacatttttatcgtaattgccctttaaaattcggggcgccgaccccatgtggtctcaagcccaaaggaagagtgaggcgagtcagtcgcgtaatgcttaggcacaggattgatttgtcgcaatgattgacacgattccgcttgacgctgcgtaaggtttttgtaattttacaggcaaccttttattcactaacaaatagctggtggaatat

SEQ ID NO: 2 - DNA, 210 bp, Escherichia coli
  source 1..210: mol_type = genomic DNA
ttaatttgatcgcccgaacagcaatgtttgggcgatttttattacgataataaagtctgtttttaatattatcatgttaaatgtttatattataaaaagtcgtttttctgcttaggattttgttatttaaattaagcctgtaatgccttgcttccattgcggataaatcctacttttttattgccttcaaataaatttaaggagttcgaa

SEQ ID NO: 3 - DNA, 371 bp, Escherichia coli
  source 1..371: mol_type = genomic DNA
tgagttattctggccgcagcctgatggacaaagcgttcattatggtgctgccggtcgcgatgtttgttgccagcggttttgagcacagtatcgcaaacatgtttatgatcccgatgggtattgtaatccgcgacttcgcatccccggaattttggaccgcagtcggttctgcaccggaaaatttttctcacctgaccgtgatgaatttcatcactgataacctgattccggttacgatcggcaacattatcggtggtggtttgttggttgggttgacatactgggtcatttacctgcgtgaaaacgaccaccattaatggttgtcgaagtacgcagtaaataaaaaatccacttaagaaggtaggtgttac

SEQ ID NO: 4 - DNA, 187 bp, Escherichia coli
  source 1..187: mol_type = genomic DNA
ttgccagttccatccggagtttacttctactccacgtgatggtcacccgctgtttgcaggctttgtgaaagccgccagcgagttccagaaacgtcaggcgaagtaagtaaaaaagttagagcggcaacgcgtaccctgggtacgcgttgtttgtctggagtttcagtttaactagtgacttgaggaa

SEQ ID NO: 5 - DNA, 184 bp, Escherichia coli
  source 1..184: mol_type = genomic DNA
caatctaccggttattttgtaaaccgtttgtgtgaaacaggggtggcttatgccgccccttattccatcttgcatgtcattatttcccttctgtatatagatatgctaaatccttacttccgcatattctctgagcgggtatgctacctgttgtatcccaatttcatacagttaaggacaggcc

SEQ ID NO: 6 - DNA, 1159 bp, synthetic construct
  source 1..1159: mol_type = other DNA
ggtgcgcataatgtatattatgttaaatcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtggtgcgcataatgtatattatgttaaat

SEQ ID NO: 7 - DNA, 21 bp, synthetic construct
  source 1..21: mol_type = other DNA
cctgaaaaacacccgtctttg

SEQ ID NO: 8 - DNA, 20 bp, synthetic construct
  source 1..20: mol_type = other DNA
atgacgcaatctcgattgca

SEQ ID NO: 9 - DNA, 20 bp, synthetic construct
  source 1..20: mol_type = other DNA
atgtcagccaaagtatgggt

SEQ ID NO: 10 - DNA, 29 bp, synthetic construct
  source 1..29: mol_type = other DNA
attggatccataaacgtaaatgaacgttc

SEQ ID NO: 11 - DNA, 20 bp, synthetic construct
  source 1..20: mol_type = other DNA
ataaacgtaaatgaacgttc

SEQ ID NO: 12 - DNA, 35 bp, synthetic construct
  source 1..35: mol_type = other DNA
tcgagattgcgtcatatattccaccagctatttgt

SEQ ID NO: 13 - DNA, 35 bp, synthetic construct
  source 1..35: mol_type = other DNA
attggatcccccggggcagtaaacgacccgtaaat

SEQ ID NO: 14 - DNA, 38 bp, synthetic construct
  source 1..38: mol_type = other DNA
ccatactttggctgacatatattccaccagctatttgt

SEQ ID NO: 15 - DNA, 35 bp, synthetic construct
  source 1..35: mol_type = other DNA
tcgagattgcgtcatttcgaactccttaaatttat

SEQ ID NO: 16 - DNA, 35 bp, synthetic construct
  source 1..35: mol_type = other DNA
attggatcccccgggttaatttgatcgcccgaaca

SEQ ID NO: 17 - DNA, 38 bp, synthetic construct
  source 1..38: mol_type = other DNA
ccatactttggctgacatttcgaactccttaaatttat

SEQ ID NO: 18 - DNA, 35 bp, synthetic construct
  source 1..35: mol_type = other DNA
tcgagattgcgtcatgtaacacctaccttcttaag

SEQ ID NO: 19 - DNA, 36 bp, synthetic construct
  source 1..36: mol_type = other DNA
attggatcccccgggtgagttattctggccgcagcc

SEQ ID NO: 20 - DNA, 38 bp, synthetic construct
  source 1..38: mol_type = other DNA
ccatactttggctgacatgtaacacctaccttcttaag

SEQ ID NO: 21 - DNA, 35 bp, synthetic construct
  source 1..35: mol_type = other DNA
tcgagattgcgtcatttcctcaagtcactagttaa

SEQ ID NO: 22 - DNA, 35 bp, synthetic construct
  source 1..35: mol_type = other DNA
attggatcccccgggttgccagttccatccggagt

SEQ ID NO: 23 - DNA, 38 bp, synthetic construct
  source 1..38: mol_type = other DNA
ccatactttggctgacatttcctcaagtcactagttaa

SEQ ID NO: 24 - DNA, 35 bp, synthetic construct
  source 1..35: mol_type = other DNA
tcgagattgcgtcatggcctgtccttaactgtatg

SEQ ID NO: 25 - DNA, 35 bp, synthetic construct
  source 1..35: mol_type = other DNA
attggatcccccgggcaatctaccggttattttgt

SEQ ID NO: 26 - DNA, 38 bp, synthetic construct
  source 1..38: mol_type = other DNA
ccatactttggctgacatggcctgtccttaactgtatg

SEQ ID NO: 27 - DNA, 38 bp, synthetic construct
  source 1..38: mol_type = other DNA
tcgagattgcgtcatactttaactctcctggattggtc

SEQ ID NO: 28 - DNA, 35 bp, synthetic construct
  source 1..35: mol_type = other DNA
attggatcccccgggaactttgcctcaggcattag

SEQ ID NO: 29 - DNA, 41 bp, synthetic construct
  source 1..41: mol_type = other DNA
ccatactttggctgacatactttaactctcctggattggtc

SEQ ID NO: 30 - DNA, 37 bp, synthetic construct
  source 1..37: mol_type = other DNA
tcgagattgcgtcatggtgaatcctctcgttgattct

SEQ ID NO: 31 - DNA, 35 bp, synthetic construct
  source 1..35: mol_type = other DNA
attggatcccccgggtacggaattgccgttggtct

SEQ ID NO: 32 - DNA, 40 bp, synthetic construct
  source 1..40: mol_type = other DNA
ccatactttggctgacatggtgaatcctctcgttgattct

SEQ ID NO: 33 - DNA, 37 bp, synthetic construct
  source 1..37: mol_type = other DNA
tcgagattgcgtcatttgctacctcctttattatcgt

SEQ ID NO: 34 - DNA, 35 bp, synthetic construct
  source 1..35: mol_type = other DNA
attggatcccccgggactgcgggctactgccctat

SEQ ID NO: 35 - DNA, 40 bp, synthetic construct
  source 1..40: mol_type = other DNA
ccatactttggctgacatttgctacctcctttattatcgt

SEQ ID NO: 36 - DNA, 36 bp, synthetic construct
  source 1..36: mol_type = other DNA
tcgagattgcgtcataatgctctcctgataatgtta

SEQ ID NO: 37 - DNA, 36 bp, synthetic construct
  source 1..36: mol_type = other DNA
attggatcccccgggttttcacctcctaactactta

SEQ ID NO: 38 - DNA, 39 bp, synthetic construct
  source 1..39: mol_type = other DNA
ccatactttggctgacataatgctctcctgataatgtta

SEQ ID NO: 39 - DNA, 35 bp, synthetic construct
  source 1..35: mol_type = other DNA
tcgagattgcgtcatgactacctctgaactttgga

SEQ ID NO: 40 - DNA, 35 bp, synthetic construct
  source 1..35: mol_type = other DNA
attggatcccccgggtcagtataaaagagagccag

SEQ ID NO: 41 - DNA, 38 bp, synthetic construct
  source 1..38: mol_type = other DNA
ccatactttggctgacatgactacctctgaactttgga

SEQ ID NO: 42 - DNA, 35 bp, synthetic construct
  source 1..35: mol_type = other DNA
tcgagattgcgtcatagctcctccttaatgacagg

SEQ ID NO: 43 - DNA, 35 bp, synthetic construct
  source 1..35: mol_type = other DNA
attggatcccccgggaactgtttgctgaagatgat

SEQ ID NO: 44 - DNA, 38 bp, synthetic construct
  source 1..38: mol_type = other DNA
ccatactttggctgacatagctcctccttaatgacagg

SEQ ID NO: 45 - DNA, 37 bp, synthetic construct
  source 1..37: mol_type = other DNA
tcgagattgcgtcatagtgtattccttattgttgctt

SEQ ID NO: 46 - DNA, 35 bp, synthetic construct
  source 1..35: mol_type = other DNA
attggatcccccgggaagccacttgcgacgacgtt

SEQ ID NO: 47 - DNA, 40 bp, synthetic construct
  source 1..40: mol_type = other DNA
ccatactttggctgacatagtgtattccttattgttgctt

SEQ ID NO: 48 - DNA, 35 bp, synthetic construct
  source 1..35: mol_type = other DNA
tcgagattgcgtcattcctggcacctcttttgtta

SEQ ID NO: 49 - DNA, 35 bp, synthetic construct
  source 1..35: mol_type = other DNA
attggatcccccgggtggcaaaggcatatgctaaa

SEQ ID NO: 50 - DNA, 38 bp, synthetic construct
  source 1..38: mol_type = other DNA
ccatactttggctgacattcctggcacctcttttgtta

SEQ ID NO: 51 - DNA, 35 bp, synthetic construct
  source 1..35: mol_type = other DNA
tcgagattgcgtcatttacccaatcctgtcttttg

SEQ ID NO: 52 - DNA, 34 bp, synthetic construct
  source 1..34: mol_type = other DNA
attggatcccccgggtctgacgggggaacctccc

SEQ ID NO: 53 - DNA, 38 bp, synthetic construct
  source 1..38: mol_type = other DNA
ccatactttggctgacatttacccaatcctgtcttttg

SEQ ID NO: 54 - DNA, 35 bp, synthetic construct
  source 1..35: mol_type = other DNA
tcgagattgcgtcatgcagatctcctggcttgctt

SEQ ID NO: 55 - DNA, 35 bp, synthetic construct
  source 1..35: mol_type = other DNA
attggatcccccgggggatggctctctttcctgga

SEQ ID NO: 56 - DNA, 38 bp, synthetic construct
  source 1..38: mol_type = other DNA
ccatactttggctgacatgcagatctcctggcttgctt

SEQ ID NO: 57 - DNA, 37 bp, synthetic construct
  source 1..37: mol_type = other DNA
tcgagattgcgtcattttaattctccacgcttataag

SEQ ID NO: 58 - DNA, 35 bp, synthetic construct
  source 1..35: mol_type = other DNA
attggatcccccgggcaaaaagcaaagcctttgtg

SEQ ID NO: 59 - DNA, 40 bp, synthetic construct
  source 1..40: mol_type = other DNA
ccatactttggctgacattttaattctccacgcttataag

SEQ ID NO: 60 - DNA, 35 bp, synthetic construct
  source 1..35: mol_type = other DNA
tcgagattgcgtcatgtaatactccgttgactgaa

SEQ ID NO: 61 - DNA, 35 bp, synthetic construct
  source 1..35: mol_type = other DNA
attggatcccccggggttacttaatttaagtgacg

SEQ ID NO: 62 - DNA, 38 bp, synthetic construct
  source 1..38: mol_type = other DNA
ccatactttggctgacatgtaatactccgttgactgaa

SEQ ID NO: 63 - DNA, 35 bp, synthetic construct
  source 1..35: mol_type = other DNA
tcgagattgcgtcatagttctcctctcttgctgaa

SEQ ID NO: 64 - DNA, 33 bp, synthetic construct
  source 1..33: mol_type = other DNA
attggatcccccggggctggtcgatcactggaa

SEQ ID NO: 65 - DNA, 38 bp, synthetic construct
  source 1..38: mol_type = other DNA
ccatactttggctgacatagttctcctctcttgctgaa

SEQ ID NO: 66 - DNA, 37 bp, synthetic construct
  source 1..37: mol_type = other DNA
tcgagattgcgtcatggaagtacctataattgatacg

SEQ ID NO: 67 - DNA, 35 bp, synthetic construct
  source 1..35: mol_type = other DNA
attggatcccccggggcttcacctcaacttcacat

SEQ ID NO: 68 - DNA, 40 bp, synthetic construct
  source 1..40: mol_type = other DNA
ccatactttggctgacatggaagtacctataattgatacg

SEQ ID NO: 69 - DNA, 34 bp, synthetic construct
  source 1..34: mol_type = other DNA
tcgagattgcgtcattacttgctccctttgctgg

SEQ ID NO: 70 - DNA, 35 bp, synthetic construct
  source 1..35: mol_type = other DNA
attggatcccccgggttctccagcactctggagaa

SEQ ID NO: 71 - DNA, 37 bp, synthetic construct
  source 1..37: mol_type = other DNA
ccatactttggctgacattacttgctccctttgctgg